(19) United States
(12) Patent Application Publication
(10) Pub. No.: US 2013/0079676 A1
(43) Pub. Date: Mar. 28, 2013

(54) NON-INVASIVE METHOD AND DEVICE FOR DETERMINING A REFERENCE PLANE OF THE HUMAN PELVIS

(71) Applicant: Aesculap AG, Tuttlingen (DE)

(72) Inventors: Thomas Hermle, Rottweil (DE); Peter Helwig, Freiburg (DE); Ingrid Dupraz, Rietheim/Weilheim (DE); Kilian Reising, Emmendingen (DE)

(73) Assignee: AESCULAP AG, Tuttlingen (DE)

(21) Appl. No.: 13/633,221

(22) Filed: Oct. 2, 2012

Related U.S. Application Data
(63) Continuation of application No. PCT/EP2011/055461, filed on Apr. 7, 2011.

(30) Foreign Application Priority Data
Apr. 9, 2010 (DE) 10 2010 016 386

(51) Int. Cl. A61B 5/00

(52) U.S. Cl. CPC A61B 5/4538
USPC 600/587

(57) ABSTRACT

A non-invasive method for the determination of a reference plane of the human pelvis is described in which method position coordinates of three points A, B, C of the reference plane are determined non-invasively in a reference coordinate system and the reference plane is calculated from the position coordinates of the three points A, B, C, wherein position coordinates of a first point A of the reference plane are determined non-invasively by detecting a first characteristic point (A) on the pelvis, which first characteristic point (A) is located on the reference plane. Furthermore, a device for non-invasively determining a reference plane of the human pelvis is also described.

### CROSS-REFERENCE TO RELATED APPLICATIONS

This application is a continuation of international application number PCT/EP2011/055461 filed on Apr. 7, 2011 and claims the benefit of German application number 10 2010 016 386.4 filed on Apr. 9, 2010, which are incorporated herein by reference in their entirety and for all purposes.

### FIELD OF THE INVENTION

The present invention relates to a non-invasive methods for the determination of a reference plane of the human pelvis generally, and more specifically to a non-invasive method for the determination of a reference plane of the human pelvis in which method position coordinates of three points of the reference plane are determined non-invasively in a reference coordinate system and the reference plane is calculated from the position coordinates of the three points, wherein position coordinates of a first point of the reference plane are determined non-invasively by detecting a first characteristic point on the pelvis, which first characteristic point is located on the reference plane, and wherein position coordinates of a second point of the reference plane are determined non-invasively by detecting a second characteristic point of the pelvis, which second characteristic point is located on the reference plane but not on the median plane of the pelvis.

Furthermore, the present invention relates to devices for the non-invasive determination of a reference plane of the human pelvis generally, and more specifically to a device for the non-invasive determination of a reference plane of the human pelvis, comprising a surgical navigation system for determining the position and orientation of at least one surgical device in space incorporating at least one surgical referencing device which comprises at least two marker elements and is arranged on or is connectable to the at least one surgical device, incorporating a detection device for detecting radiation emitted by the marker elements or reflected from the marker elements and also incorporating a data processing unit, wherein the actual marker coordinates of positions of the marker elements in space are determinable by the navigation system, wherein position coordinates of three points of the reference plane are determinable non-invasively in a reference coordinate system by the device and the reference plane is calculable by the data processing unit from the position coordinates of the three points, wherein an instruction to determine position coordinates of a first point of the reference plane non-invasively by detecting a first characteristic point on the pelvis, which first characteristic point is located on the reference plane, is issuable by the device and wherein an instruction to determine position coordinates of a second point of the reference plane non-invasively by detecting a second characteristic point of the pelvis, which second characteristic point is located on the reference plane but not on the median plane of the pelvis, is issuable by the device.

### BACKGROUND OF THE INVENTION

For some time, navigation systems have been employed in the field of surgery in order to assist surgeons especially when inserting implants and in the course of other operations. In general thereby, it is necessary to describe anatomical conditions in an abstract way in order to capture them in the form of mathematical models. In connection therewith, the anatomical conditions can be recorded by a navigation system and the data obtained in this manner can be processed with the aid of a data-processing system in order to assist the surgeon in selecting an implant of optimal size and inserting it into the body of the patient at the correct position and with the correct orientation for example.

From DE 20 2005 003 317 A1 for example, it is known to describe the pelvic bone by a reference plane which is defined as the pelvic inlet plane. Hereby, the pelvic inlet plane is defined by three salient points of the pelvic bone which are also referred to as the characteristic points thereof, namely the following points:

Point A: the pubic bone (symphysis pubis)

Point B: the right front iliac spine (right spina iliaca anterior superior)

Point C: the left front upper iliac spine (left spina iliaca anterior superior)

These characteristic points can, for example, be felt percutaneously but can also be determined by image forming methods.

Independently of the way in which the aforementioned characteristic points are determined however, they can be used in order to describe the pelvic inlet plane so that the latter can be used as a reference plane for the calculation of the position of the pelvic bone in subsequent calculations, for example, in regard to the selection of an implant as well as the positioning and orientation thereof relative to the pelvic bone.

A prerequisite for this method however is that at least three characteristic points which are located on the reference plane should also be ascertainable, for example, by percutaneous touch or by the use of an image forming method, preferably by representation of the pelvic bone by means of a navigated ultrasonic sensor. However it cannot be ensured in every operation that three points which are located on the reference plane will be ascertainable, and especially not when a patient is positioned on his side on an operating table. In the event of such a side position, either only the left or the right front upper iliac spine is capable of being reached.

A method for the determination of the pelvic inlet plane of the pelvic bone for the case where a patient is positioned on his side on an operating table and only one of the two front upper iliac spines is detectable has already been proposed in DE 10 2007 049 671 A1. This method envisages the determination of the distance vector between two further characteristic points of the pelvis, namely the two rear upper iliac spines and then to determine the non-accessible front upper iliac spine in a virtual manner by setting this vector outgoing from the accessible front upper iliac spine, wherein the vector is extended additionally by a defined factor. However, this defined factor or aspect ratio factor can vary significantly from patient to patient. In particular, there are differences for this value between men and women. For the practical implementation of the method, it is necessary here in particular to have empirical items of data which serve to provide a default value of the aspect ratio factor for the extension of the vector defined between the rear upper iliac spines. Thus, without such data, the accuracy of the method comes into question.

Consequently, it would be desirable to provide a method and a device of the type described hereinabove which allow the determination of a reference plane of the human pelvis with high accuracy and in particular without additional empirical data.

### SUMMARY OF THE INVENTION

In a first aspect of the invention, a non-invasive method for the determination of a reference plane of the human pelvis in which method position coordinates of three points A, B, C of the reference plane are determined non-invasively in a reference coordinate system and the reference plane is calculated from the position coordinates of the three points A, B, C. Position coordinates of a first point A of the reference plane are determined non-invasively by detecting a first characteristic point (A) on the pelvis, which first characteristic point (A) is located on the reference plane. Position coordinates of a second point (B, C) of the reference plane are determined non-invasively by detecting a second characteristic point (B; C) of the pelvis, which second characteristic point (B; C) is located on the reference plane but not on the median plane of the pelvis. The position of the median plane of the pelvis in the reference coordinate system is determined non-invasively and position coordinates of a third point (C; B) of the reference plane are calculated by mirroring the second point (B; C) of the reference plane on the median plane.

In a second aspect of the invention, a device for the non-invasive determination of a reference plane of the human pelvis, comprises a surgical navigation system for determining the position and orientation of at least one surgical device in space incorporating at least one surgical referencing device which comprises at least two marker elements and is arranged on the at least one surgical device or is connectable thereto, incorporating a detection device for detecting radiation emitted by the marker elements or reflected by the marker elements and also incorporating a data processing unit. The actual marker coordinates of positions of the marker elements in space are determinable by the navigation system. Position coordinates of three points A, B, C of the reference plane in a reference coordinate system are determinable non-invasively by the device and the reference plane is calculable from the position coordinates of the three points A, B, C by the data processing unit. An instruction is issuable by the device to determine position coordinates of a first point A of the reference plane non-invasively by detecting a first characteristic point A on the pelvis, which first characteristic point A is located on the reference plane. An instruction is issuable by the device to determine position coordinates of a second point B, C of the reference plane non-invasively by detecting a second characteristic point (B; C) of the pelvis, which second characteristic point (B; C) is located on the reference plane but not on the median plane of the pelvis. The position of the median plane of the pelvis in the reference coordinate system is determinable non-invasively by the device and wherein the data processing unit is configured and programmed such that position coordinates of a third point (C; B) of the reference plane are calculable from the position coordinates of the second point (B; C) of the reference plane by mirroring on the determined median plane.

### BRIEF DESCRIPTION OF THE DRAWING FIGURES

The foregoing summary and the following description may be better understood in conjunction with the drawing figures, of which:

FIG. 1: shows a schematic illustration of a device for non-invasively determining a reference plane of the human pelvis;

FIG. 2: a schematic illustration of the human pelvis with three characteristic points spanning the pelvic inlet plane;

FIG. 3: a schematic illustration of the lower region of the spinal column with marked characteristic points on the sacral bone as well as the lower lumbar spine which are located on the median plane; and

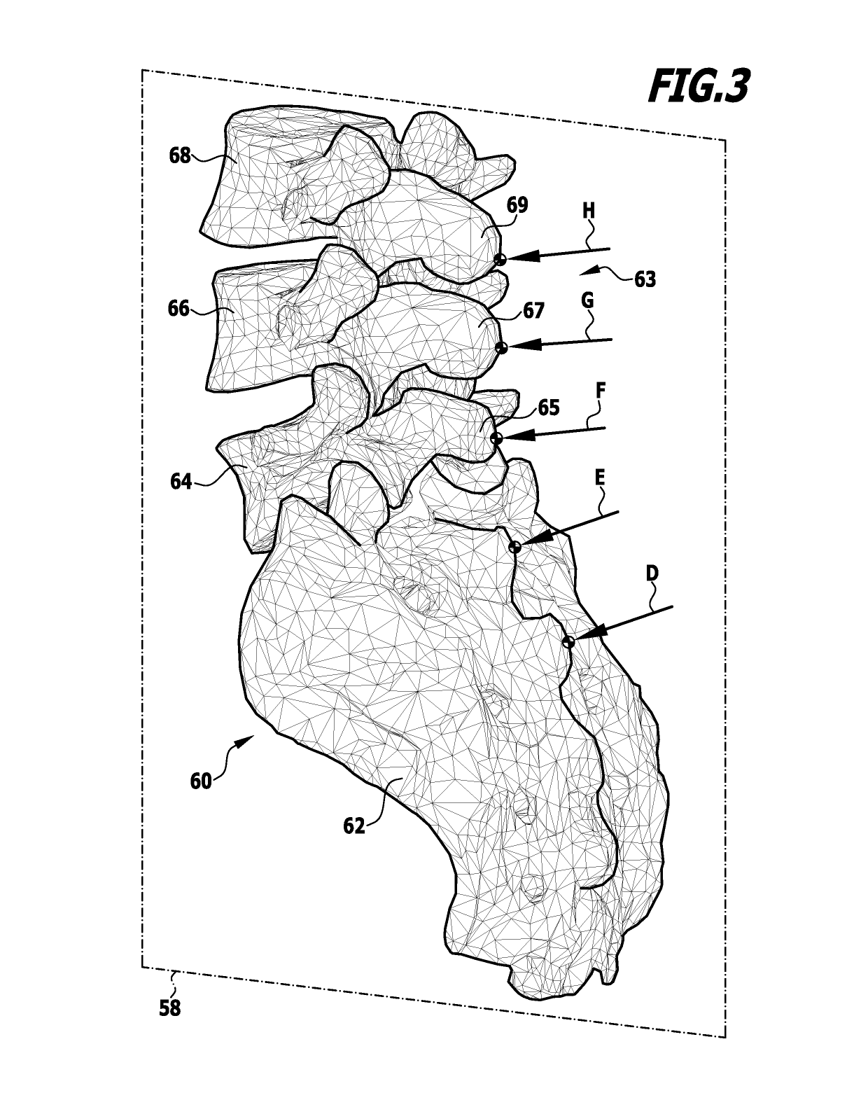

FIG. 4: a schematic plan view of a pelvic bone with marked salient points thereof.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a non-invasive method for the determination of a reference plane of the human pelvis in which method position coordinates of three points A, B, C of the reference plane are determined non-invasively in a reference coordinate system and the reference plane is calculated from the position coordinates of the three points A, B, C, wherein position coordinates of a first point A of the reference plane are determined non-invasively by detecting a first characteristic point (A) on the pelvis, which first characteristic point (A) is located on the reference plane, and wherein position coordinates of a second point (B, C) of the reference plane are determined non-invasively by detecting a second characteristic point (B; C) of the pelvis, which second characteristic point (B; C) is located on the reference plane but not on the median plane of the pelvis, wherein the position of the median plane of the pelvis in the reference coordinate system is determined non-invasively and wherein position coordinates of a third point (C; B) of the reference plane are calculated by mirroring the second point (B; C) of the reference plane on the median plane.

The method proposed according to the invention takes advantage of the fact that the human pelvis is substantially symmetrical. If at least one characteristic point of the pelvis, which point is not located on the median plane and is spaced from the median plane defining a mirror plane of the pelvic bone, is determined, then, due to the symmetry of the pelvis, a point that is likewise located on the reference plane can be easily calculated—at least in the ideal case—in that the determined characteristic point, the second characteristic point of the pelvis defined above for example, is mirrored on the non-invasively determined median plane and so a third point of the reference plane is calculated virtually. The special feature here is that if the median plane is determined directly by determining the position coordinates of points which are located on the median plane then it is not necessary to draw upon empirical data regarding the pelvic bone of a human being. Consequently, the reference plane of the pelvis, the pelvic inlet plane described hereinabove for example, can be determined to a very high degree of accuracy. Thus, the method that is proposed in accordance with the invention is not only simpler to implement than the method known from DE 10 2007 049 671 A1 but in addition to that it is also significantly more precise.

It is expedient if the pelvic inlet plane is designated as the reference plane. In this way, values, which are determined by the method proposed in accordance with the invention, can be compared with values which define the pelvic inlet plane that have been determined by other methods.

It is advantageous if, for the non-invasive determination of the median plane, position coordinates of at least three characteristic points on the pelvis and/or on bone parts adjacent to the pelvis in the reference system, which at least three characteristic points are located on the medial plane, are determined by non-invasive detection of the points, and if the median plane in the reference system is calculated from the position coordinates of the at least three characteristic points. In this way, the median plane in particular can be determined directly by detecting three salient points which themselves are located on the median plane due to the symmetrical construction of the pelvis. Complicated calculations and empirical observations for the purposes of determining the median plane are thereby redundant.

The method is particularly simple to implement if one of the at least three characteristic points on the pelvis is selected in such a way that it is located on both the median plane and on the reference plane. This manner of procedure makes it possible to determine both the reference plane and the median plane non-invasively using a total of just five detected points.

Expediently, the first characteristic point is selected in such a way that it forms the one of the at least three characteristic points on the pelvis. If, for example, this first characteristic or salient point is detected, then one has already determined one point of the reference plane and one point of the median plane in a single detection process.

In order to enable the reference plane to be determined with a particularly high degree of accuracy, it is advantageous for a point of the pelvis which is located as far away as possible from the median plane to be selected as the second characteristic point.

In principle, it would be possible to determine the position coordinates of the characteristic points of the pelvis in any coordinate system, for example, in the preferred system of the space in which the method is being carried out. Since, in the case of a surgical intervention in particular, one cannot exclude the possibility of a patient moving, it is expedient for a coordinate system that is fixed relative to the pelvis to be selected as the reference coordinate system. In this case, it is even possible for the patient's bed to be changed completely, i.e. in particular to move to another location, whereby the reference coordinate system nevertheless remains the same.

A reference coordinate system can be provided in a particularly simple way, if the fixed coordinate system is defined by a reference element that is fixed to the pelvis. For example, a reference element which is detectable by the navigation system can be fixed to the pelvis in an invasive or non-invasive manner. Then, for example, all the position coordinates of the characteristic points that have been determined with the help of the navigation system can be expressed with reference to the localised reference element fixed to the pelvis.

It is expedient if the process of detecting the characteristic points on the pelvis and/or on bone parts adjacent to the pelvis is carried out non-invasively by means of a percutaneous process of palpation thereof using a navigated palpation instrument and/or by ultrasonic scanning using a navigated ultrasonic sensor. For example, the navigated ultrasonic sensor may be an ultrasonic head of an ultrasonic apparatus with which bone structures detected by the ultrasonic sensor are presentable and measurable by means of an image forming process. In this case, “navigated” means in particular that, for the purposes of detecting the characteristic points, the navigated palpation instrument and/or the ultrasonic sensor are equipped or arranged to be equipped with a reference element, the position and orientation in space of which are determinable by means of a navigation system. Thus, for example, the position coordinates of the characteristic points can be determined very precisely from an ultrasonic image or from a point sampled by the palpation instrument which is defined by a tip of the palpation instrument for example.

Preferably, the pubic bone (symphysis pubis) is detected as the first characteristic point, the pubic bone is by definition located on the median plane of the pelvis. Particularly in the case of the pelvic inlet plane, the pubic bone is also located thereon so that, by detecting the position of the pubic bone, one point of the reference plane and one point of the median plane are already ascertained.

It is expedient for the three characteristic points defining the reference plane to be located as far apart as possible. Consequently, it is expedient if a characteristic point on the iliac crest is detected non-invasively as the second characteristic point.

It is advantageous if it is detected non-invasively as one of the two front upper iliac spines (spina iliaca anterior superior). This marked point can be detected in a particularly certain and easy manner. As a rule, only one of these two front upper iliac spines can be detected when a patient is lying on his side.

Since the other one of the two front upper iliac spines is not detectable in this side position but should nevertheless be used for the optimal determination of the reference plane, it is advantageous for the other one of the non-palpated front upper iliac spines (spina iliaca anterior superior) to be selected as the third point of the reference plane and for the position coordinates thereof to be calculated. The calculation is effected in the manner described in accordance with the invention by mirroring the position coordinates of the palpated front upper iliac spine on the median plane. In this way, the non-palpated front upper iliac spine, or stated more precisely, the position coordinates thereof, can be determined in a virtual manner.

It is advantageous, if a characteristic point of the pelvis which is located on the median plane but not on the reference plane is detected non-invasively as one of the at least two further characteristic points. Preferably, the point of the median plane which is not located on the reference plane and which is to be detected is located as far away as possible from the first characteristic point.

Such a characteristic point of the pelvis can be detected in a particularly simply and certain manner, if at least one tubercle of the sacral bone is detected non-invasively as such a point. The designation tubercle is used in the following as a synonym for a vertebral spinous process, for example, of the sacral bone or of a lumbar vertebra.

Since the sacral bone is usually easily accessible even if a patient is lying in a side position on an operating table, it is advantageous if two tubercles of the sacral bone are detected non-invasively as two characteristic points of the pelvis. These are located on the median plane due to the shape of the pelvis itself. In particular, if the pelvic inlet plane defined hereinabove is intended to be specified as the reference plane, it is ensured with a very high degree of probability that the two tubercles or vertebral spinous processes of the sacral bone are not located on the reference plane, whereby this would otherwise also be irrelevant.

Furthermore, on the assumption that there is practically no lateral displacement between the lower lumbar vertebrae and the sacral bone even in a side position, it can be expedient if a characteristic point of the lower lumbar spine which is located on the median plane is detected non-invasively as one of the at least three further characteristic points.

One of these characteristic points can be detected in a particularly simple and certain manner, if at least one tubercle on one of the first three lumbar vertebrae is detected non-invasively as the characteristic point of the lower lumbar spine. Such a tubercle can be defined in particular as a salient projection in the form of a vertebral spinous process which projects away from a lumbar vertebra in the posterior direction. In particular, the detection process can be effected on the lumbar vertebrae L5, L4 and L3.

Furthermore, it can be advantageous if that point which is located at the centre of a line connecting the two rear upper iliac spines (spina iliaca posterior superior) is selected as one of the at least three further characteristic points. Ideally, the rear upper iliac spines are arranged symmetrically with respect to the median plane so that the point located centrally therebetween is then inevitably located on the median plane.

In general however, this point is not detectable directly. Consequently, it is advantageous if the position coordinates of the point which is located at the centre of the line connecting the two rear upper iliac spines are calculated from the position coordinates of the two rear upper iliac spines that were determined by a non-invasive detection process. For example, the point can be determined in that the position vector from the left rear upper iliac spine to the right rear upper iliac spine is determined and commencing from one of the two, the half length of the vector that has been determined in this way is added to the position vector of this iliac spine. The point determined in this way is then quasi a virtual point of the pelvis which is defined by two non-invasively detectable salient or characteristic points of the same.

Further, the present invention relates to a device for the non-invasive determination of a reference plane of the human pelvis, comprising a surgical navigation system for determining the position and orientation of at least one surgical device in space incorporating at least one surgical referencing device which comprises at least two marker elements and is arranged on the at least one surgical device or is connectable thereto, incorporating a detection device for detecting radiation emitted by the marker elements or reflected by the marker elements and also incorporating a data processing unit, wherein the actual marker coordinates of positions of the marker elements in space are determinable by the navigation system, wherein position coordinates of three points A, B, C of the reference plane in a reference coordinate system are determinable non-invasively by the device and the reference plane is calculable from the position coordinates of the three points A, B, C by the data processing unit, wherein an instruction is issuable by the device to determine position coordinates of a first point A of the reference plane non-invasively by detecting a first characteristic point A on the pelvis, which first characteristic point A is located on the reference plane, and wherein an instruction is issuable by the device to determine position coordinates of a second point B, C of the reference plane non-invasively by detecting a second characteristic point (B; C) of the pelvis, which second characteristic point (B; C) is located on the reference plane but not on the median plane of the pelvis, wherein the position of the median plane of the pelvis in the reference coordinate system is determinable non-invasively by the device and wherein the data processing unit is configured and programmed such that position coordinates of a third point (C; B) of the reference plane are calculable from the position coordinates of the second point (B; C) of the reference plane by mirroring on the determined median plane.

As already expressed in detail above, with the aid of such a device it is possible in a simple manner to determine a reference plane of the human pelvis such as the pelvic inlet plane that was defined hereinabove for example. With the help of the navigation system, all of the points necessary for the determination of the median plane can be detected, the median plane calculated and, by mirroring the position coordinates of one of the ascertained characteristic points on the calculated median plane, a third characteristic point which is not accessible for a direct determination process due to the position of the person, for example when the latter is positioned on his side, can be calculated virtually.

It is advantageous, if the device is constructed in such a manner that the pelvic inlet plane is determined as the reference plane. This manner of procedure offers the option of comparing the pelvic inlet plane determined by the device in accordance with the invention with pelvic inlet planes determined by means of other methods in order to thus verify the accuracy of the method for example.

In accordance with a further preferred embodiment of the invention for the purposes of determining the median plane in a non-invasive manner, provision may be made for one or more instructions to be issuable by the device for determining position coordinates of at least three characteristic points on the pelvis and/or on bone parts adjacent to the pelvis in the reference system, which at least three characteristic points are located on the median plane, by non-invasive detection of the points, and in that the data processing unit is configured and programmed such that the median plane in the reference system is calculated from the position coordinates of the at least three characteristic points. The device thus enables a person to be guided when implementing the method described above and, after detection of the characteristic points necessary for the calculation of the reference plane, to firstly calculate the median plane and then too the reference plane by means of this median plane.

It is expedient if an instruction is issuable by the device that one of the at least three characteristic points on the pelvis is to be selected in such a way that it is located both on the median plane and on the reference plane. This simplifies the determination of the reference plane significantly, since, by the choice of such a point, a total of just five characteristic points have to be detected in order to determine both the median plane and the reference plane.

Preferably, an instruction is issuable by the device that the first characteristic point is to be selected in such a way that it forms the one of the at least three characteristic points on the pelvis. In other words, this means that the first characteristic point which is located on the reference plane should also be located on the median plane at the same time. Thus, by the detection of just a single point, a point of one of the two planes, i.e. the median plane and the reference plane is determined automatically.

It is expedient if an instruction is issuable by the device that a point of the pelvis which is located as far away as possible from the median plane be selected as the second characteristic point. The choice of such a point can, in particular, have the advantage that the reference plane is determinable to a very high degree of accuracy.

In accordance with a further preferred embodiment of the invention, provision may be made for the navigation system to be configured and programmed such that a coordinate system which is fixed relative to the pelvis is selected as the reference coordinate system. With the aid of such a device, the pelvic inlet plane can be determined in a certain and highly precise manner even if the person whose pelvis is to be measured should move during the process of detecting the characteristic points.

Advantageously, the device comprises a reference element which is fixable to the pelvis or to a fixing element, in the form of a bone screw for example, and the position of which in space is detectable by the navigation system. Such a reference element makes it possible to define a coordinate system that is fixed relative to the pelvis in a simple manner. In consequence, all the position coordinates of the detected characteristic points can be determined in this coordinate system that is fixed relative to the pelvis by means of the navigation system.

It is further expedient if the device comprises a position detection device with which, in conjunction with the navigation system, position coordinates of the characteristic points on the pelvis and/or on the bone parts adjacent to the pelvis are determinable in a non-invasive manner. The position detection device may, in particular, be a surgical device which carries a referencing device comprising at least two marker elements, or which is connectable to such a referencing device. In consequence, the position and the orientation of the position detection device in the reference coordinate system for example can be determined by the navigation system.

The characteristic points can be detected in the described way in a particularly simple and certain manner if the position detection device is in the form of a navigable palpation instrument and/or in the form of a navigable ultrasonic sensor, for example, both the palpation instrument and the ultrasonic sensor may carry a reference element or be connectable to such an element, the position and orientation in space of which are determinable by the navigation system in order to determine indirectly the position coordinates of the characteristic points that are to be determined by the measurement of the position and the orientation of the position detection device in space.

It is expedient if an instruction is issuable by the device that the pubic bone (symphysis pubis) is to be detected as the first characteristic point. An operator can thus be guided by the device into detecting a first characteristic point of the pelvis in order to determine its position coordinates.

Furthermore, it can be expedient if an instruction is issuable by the device that one of the two front upper iliac spines (spina iliaca anterior superior) be detected non-invasively as the second characteristic point. In particular, the message can be sent that it is the front upper iliac spine facing the operator that is detected. The front upper iliac spine remote from the operator is generally not accessible especially when the patient is positioned on his side. This point can then be determined virtually by the device in accordance with the invention by mirroring the front upper iliac spine facing the operator on the median plane.

It is especially advantageous if the device is configured and programmed such that the other undetected front upper iliac spine (spina iliaca anterior superior) is virtually determinable by the data processing unit as the third point of the reference plane by a process of calculating its position coordinates by mirroring the position coordinates of the detected front upper iliac spine on the calculated median plane. After the detection of the first and second characteristic points as well as the points necessary for the determination of the median plane, the third point of the reference plane can be determined virtually with the aid of the third point of the reference plane that has been determined in such a manner and thus too the position of the reference plane in the reference coordinate system, for example, in the fixed coordinate system of the pelvis.

It is expedient if an instruction is issuable by the device that a characteristic point of the pelvis which is located on the median plane but not on the reference plane be detected non-invasively as one of the at least two further characteristic points. Configuring the device in such a manner has the advantage that it can be ensured in this way that at least one of three characteristic points spanning the median plane is not located on the reference plane. The median plane can thus be determined in a definite manner in order to be used in the further calculation for the reference plane that is to be determined.

It is advantageous if an instruction is issuable by the device that at least one tubercle of the sacral bone be detected non-invasively as a characteristic point of the pelvis. Due to the position of the tubercle of the sacral bone on the median plane, a characteristic point thereof can thus be detected in a simple manner.

It can be expedient, furthermore, if an instruction is issuable by the device that two tubercles of the sacral bone be detected non-invasively as two characteristic points of the pelvis. The tubercles of the sacral bone are normally easily accessible even in a side disposition of the patient and, in conjunction with a further point on the pelvis which is located on the median plane and has been detected, enable the determination of the position of the median plane in the reference coordinate system to be effected directly.

Preferably, an instruction is issuable by the device that at least one tubercle on one of the first three lumbar vertebrae be detected non-invasively as a characteristic point of the lower lumbar spine. For example, it can be characteristic points of the first lumbar vertebrae which are freely accessible even in a side disposition. For example, it may be a characteristic or salient projection on a vertebral spinous process of the lumbar vertebrae L5, L4 or L3. At this point furthermore, it should be pointed out that more than three characteristic or salient points on the pelvis and/or on the adjacent lumbar spine can be detected for the purposes of determining the median plane. It is true that by detecting four or more points the median plane will be over-specified, but any inaccuracies in the detection of the individual points can then be avoided by adapting the optimum median plane which is least distant from all the points that have been detected.

Furthermore, in accordance with a further preferred embodiment of the invention, provision may be made for the device to be configured and programmed in such a manner that that point which is located at the centre of a line connecting the two rear upper iliac spines (spina iliaca posterior superior) is virtually determinable as one of the at least three further characteristic points. By taking into consideration the fact that the pelvis is symmetrical with respect to the median plane, the described point is likewise located on the median plane. It can thus be calculated by the detection of two characteristic points of the pelvis that are located such as to be mutually mirror-symmetrical with respect to the median plane. Moreover, a further option for the determination of the median plane consists in that, in addition to this characteristic point at the centre of the line connecting the two rear upper iliac spines, just one further point located on the median plane is determined and the median plane that is to be calculated is placed perpendicularly to the line connecting the two rear upper iliac spines through the centre thereof and the further determined point of the median plane. Nevertheless, over-specification is again a result of this manner of procedure. In principle, it would be sufficient to put the median plane perpendicularly through the point at the centre of the line connecting the two rear upper iliac spines and perpendicularly to the connecting line. It follows therefore that the detection of an additional point can be used to increase the accuracy with which the median plane is determined.

It is advantageous if the device is configured and programmed in such a manner that the position coordinates of the point which is located at the centre of the line connecting the two rear upper iliac spines are calculated by the data processing unit from the position coordinates of the two rear iliac spines that are determined by a non-invasive detection process. The position of the median plane in the reference coordinate system can thus be calculated by means of the data processing unit with the aid of the position coordinates and possibly further determined position coordinates of characteristic points of the pelvis which are located on the median plane.

Furthermore, the invention relates to a computer program product comprising a computer-readable medium and a computer program stored on the computer-readable medium having program code means which are suitable for implementing one of the methods described above when the computer program is running on a computer of a navigation system. In this connection, it should be noted that the program is preferably configured such that instructions are issued by the computer of the navigation system to detect the points that are to be determined. The sequence in which the points that are to be detected are determined is not relevant in the present case and in all the methods described above.

Moreover, the invention relates to a computer program comprising program code means which are suitable for implementing one of the methods described above when the computer program is running on a computer of a navigation system. Here too, it should be noted that the computer program preferably controls the computer of the navigation system such that it issues instructions for guiding an operator when carrying out the methods described above.

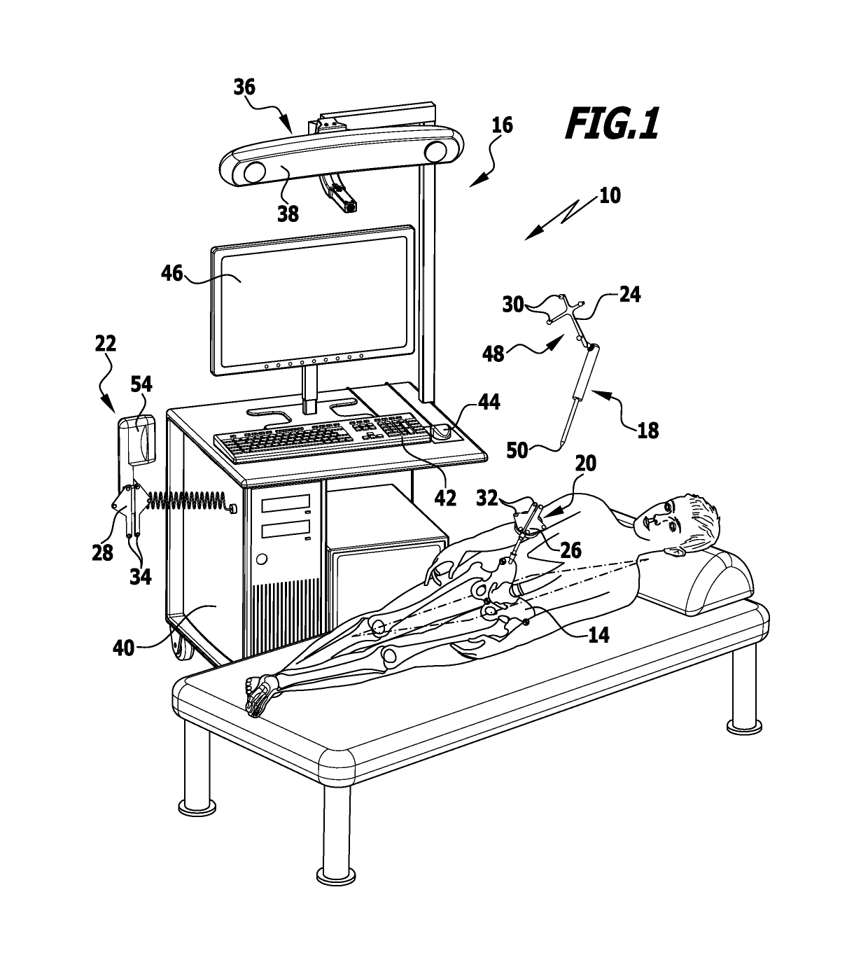

A device 10 for non-invasively determining a reference plane 12 of a human pelvis 14 is illustrated in exemplary manner in FIG. 1. It comprises a surgical navigation system 16 for determining the position and orientation of surgical devices 18, 20 and 22 in space. These devices 18, 20 and 22 each carry a respective referencing device 24, 26 and 28 which each comprise at least two passive marker elements 30, and in particular, three, four or more, or at least two active marker elements 32 and 34, whereby preferably three, four, five or six marker elements are provided for forming one of the active referencing devices 26, 28.

Furthermore, the navigation system 16 comprises a detection device 36 for detecting radiation reflected from or emitted by the marker elements 30, 32, 34. In particular, a stereo-camera 38 which can selectively detect electromagnetic radiation, and in particular infrared radiation, or ultrasonic radiation may be provided for this purpose. Furthermore, the navigation system 16 comprises a data processing unit 40 in the form of one or more computers which are operated via conventional input devices such as those in the form of a keyboard 42 or a mouse 44 for example. A display device 46 in the form of a monitor is coupled to the computer in order to display data and instructions to an operator. The actual marker coordinates of the positions of the marker elements 30, 32 and 34 in space can be determined in known manner by the navigation system 16.

The exemplarily illustrated and described surgical devices 18, 20 and 22 are of differing type. The surgical device 18 is in the form of a palpation instrument 48 which comprises a probe tip 50. If the positions of the passive marker elements 30 are determined by the navigation system 16, then the position of the probe tip 50 can be calculated from their position and orientation in space. In this way, it is possible to scan points in space with the probe tip 50, for example, characteristic points on the bones of the human body, by means of a palpation process, in particular, by contacting these points percutaneously with the probe tip 50. Self-evidently, the palpation instrument 48 could also be equipped with or be an active referencing device 26 or 28 in place of the passive referencing device 24.

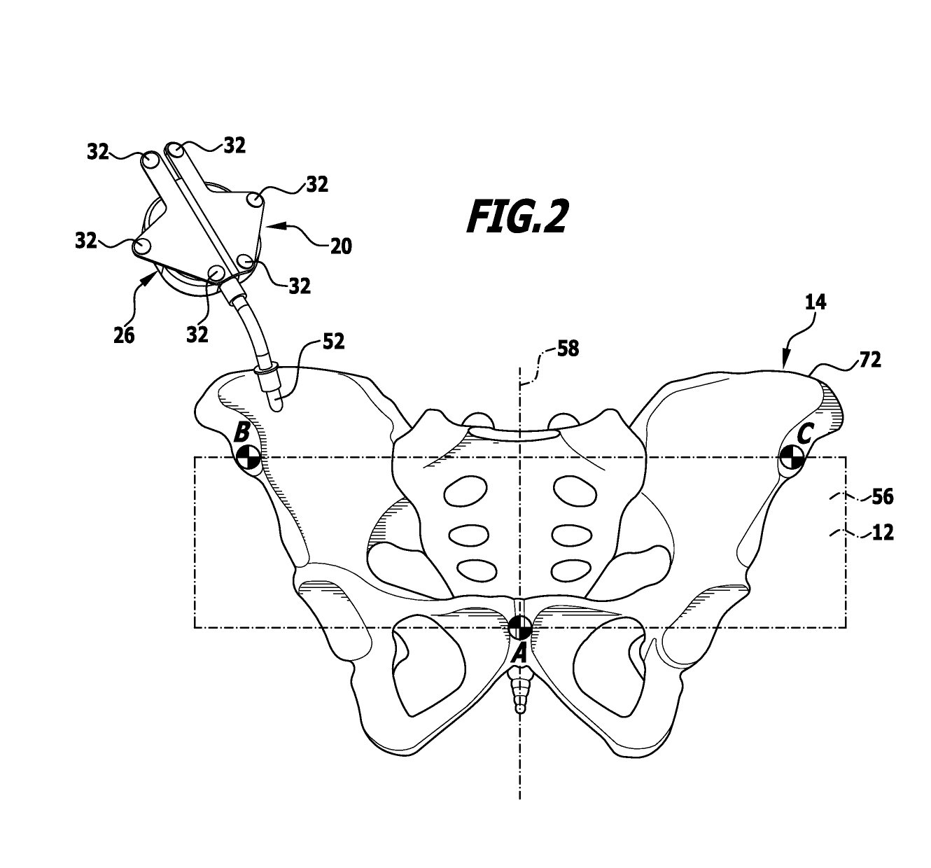

In the case of the surgical device 20, this is an active referencing device 26 which is connectable in detachable manner to, in particular, a bone screw. The referencing device 26 serves, in particular, for defining a reference coordinate system. The reference coordinate system is defined in order to specify all of the detected points in coordinates of this reference coordinate system. For example, if the referencing device 26 is fixed temporarily to the pelvis 14 as schematically illustrated in FIGS. 1 and 2, then a coordinate system that is fixed relative to the pelvis 14 can be defined in this way and all the points that are to be determined are specified in this fixed coordinate system.

The surgical device 22 comprises an ultrasonic sensor 54 which carries the referencing device 28 and with which, the body of the patient can be scanned with ultrasonic waves for example. The ultrasonic image generated thereby can, in particular, be displayed on the display device 46.

As already mentioned hereinabove, the device 10 is used especially when preparing for a surgical procedure such as the implantation of artificial joints. For example, when implanting an artificial hip joint, it is important that the alignment of the socket and shaft of a hip joint endoprosthesis relative to the pelvis 14 is effected in the desired position in order to ensure the re-establishment of the natural movement of the members concerned after a successful implantation operation. In order to enable angular positions relative to the pelvis 14 to be established here in a defined way, the reference plane 12 is used, inter alia, as a reference factor.

Preferably, the reference plane 12 is the pelvic inlet plane 56 which is spanned by the three points A, B and C that are illustrated schematically in FIG. 2. The point A is the pubic bone (symphysis pubis), the point B is the right front upper iliac spine (spina iliaca anterior superior right) and finally, the point C is the left front upper iliac spine (spina iliaca anterior superior left). Point B and point C are salient points of the iliac crest 72. The aforementioned three points A, B, C are salient or characteristic points of the pelvis 14 which can be detected selectively either percutaneously by means of the palpation instrument 48 or by the navigated ultrasonic sensor 54. In the course of the palpation process, the salient points A, B, C can then be selected and confirmed on the display device 46 by an operator for example.

If the patient is lying in the upside-down position, then, in principle, the aforementioned three points A, B and C are directly accessible. If, however, the patient is lying on an operating table such that he is positioned on his side as is schematically illustrated in FIG. 1, then only one of the front upper iliac spines is accessible. In the case of the situation illustrated in FIG. 1, this is the point B. Point C cannot be detected directly by the palpation instrument 48 available here nor the ultrasonic sensor 54.

As is being proposed in accordance with the invention, the non-accessible and thus non-directly detectable point C, or expressed more precisely, the position coordinates thereof, is calculated. To this end, the median plane 58 of the pelvis 14 is firstly determined. For this purpose for example, just three characteristic points located on the median plane 58 have to be detected with the palpation instrument 48 or the ultrasonic sensor 54. These may, for example, be the point A or further points of the pelvis 14 or of bone parts adjacent to the pelvis 14 and in particular the lumbar spine 63 of the spinal column 60. Coming into consideration in particular are the point D, namely, the first tubercle of the sacral bone 62 (tubercle sacrum) which is also referred to as a vertebral spinous process, the point E, namely, the second tubercle of the sacral bone 62 (tubercle sacrum), the point F, namely the vertebral spinous process 65 of the fifth lumbar vertebra 64 pointing in the posterior direction, the point G, namely the tubercle 67 of the fourth lumbar vertebra 66 pointing in the posterior direction, and the point H, namely the vertebral spinous process 69 of the third lumbar vertebra 68 pointing in the posterior direction. The aforementioned points A, D, E, F, G, H are all located on a mirror plane of the pelvis 14, and thus too, of the entire body of the patient, which is defined by the median plane 58.

If three of the aforementioned points A, D, E, F, G and H are selectively detected in the described way, then the median plane 58 can be calculated in a simple manner from the position coordinates of the three detected points.

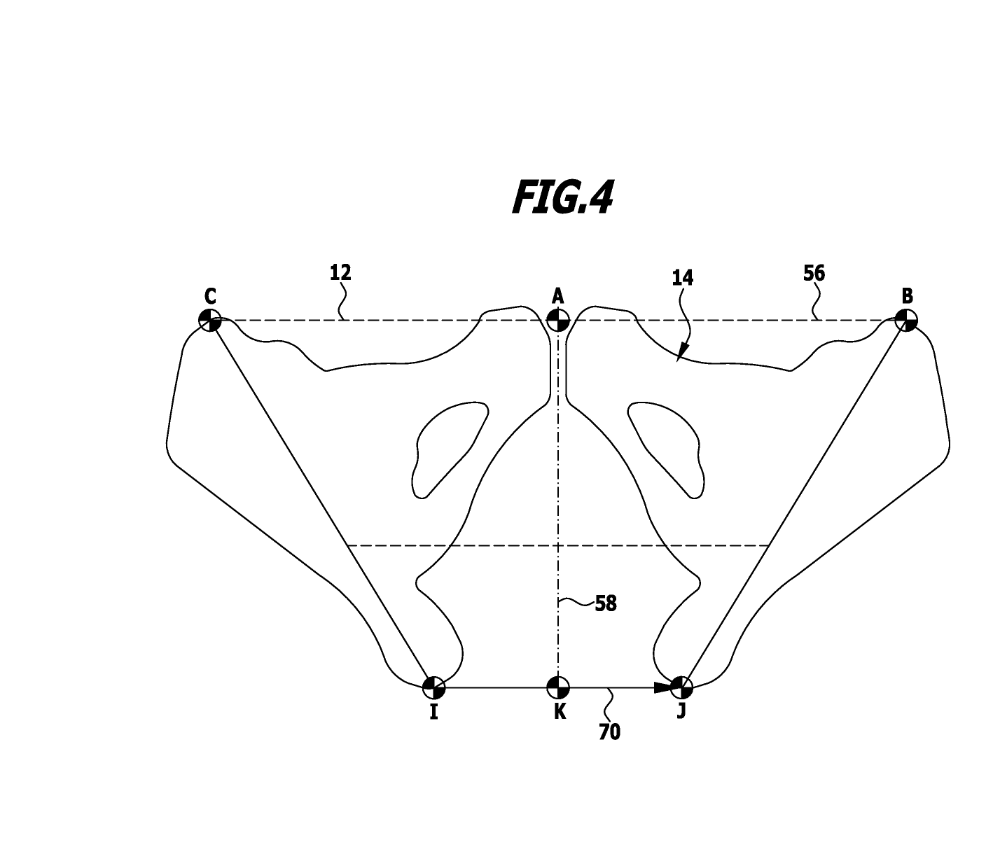

Furthermore, a further point, which is located on the median plane 58, can be determined. In connection therewith, this is the point K which is depicted schematically in FIG. 4 and is located at the centre of the line 70 connecting the point I, namely the right rear upper iliac spine (spina iliaca anterior superior right) and the point J, namely the left rear upper iliac spine (spina iliaca anterior superior left). As a rule, the rear upper iliac spines are readily detectable directly even in the side position of the patient.

An alternative possibility for determining the median plane is that, after the process of determining the point K, one positions a plane perpendicular to the connecting line 70 through the point A and the point K.

The determination of the median plane 58 is not an end in itself. By taking into consideration the highly symmetrical nature of the pelvis 14, the point C can be calculated by mirroring the detected point B on the median plane 58. Alternatively, the point B can be calculated by mirroring the position coordinates of the point C on the median plane 58 after the point C has been detected. The virtual characteristic point C or the one calculated in such a way together with the characteristic points A and B then span the pelvic inlet plane 56 which then serves as the reference plane 12 for further calculations for assisting a surgeon with the selection and implantation of a joint endoprosthesis.

Other than was the case for the known methods described hereinabove, it is now possible with the method being proposed for the median plane 58 to be determined directly by detecting three characteristic points A, D, E, F, G, H, K that are located thereon due to the symmetrical shape of the pelvis 14.

For the purposes of carrying out the method, the navigation system 16 is preferably programmed in such a manner that an operator is requested to probe for the characteristic points that are to be detected for the purposes of determining the median plane 58 as well as the reference plane 12. This can be effected by the display of an appropriate instruction in the form of a scanning request on the display device 46. The capturing of the position coordinates of the respective points then takes place in a conventional manner by a detection process using the palpation instrument 48 or the ultrasonic sensor 54 in the manner described, whereby a operator confirms, preferably by means of an appropriate input on the keyboard 42 or the mouse 44 or some other input device, that the characteristic point that is to be detected is being scanned or is visible in the desired manner at the moment of acknowledgement. This request for input is repeated by the navigation system 16 until such as time as at least the minimum number of characteristic points that are necessary for the determination of the median plane 58 and also the reference plane 12 have been detected.

Optionally the navigation system 16 can also be configured and programmed such that the respective planes, in particular the median plane 58, can be calculated in an over-determined way, i.e. that more than three characteristic points will be detected and that from these, the best fitting median plane 58 is then determined, this being the one at the least distance from all the determined characteristic points. Preferably however, the median plane 58 is selected in such a way that the point A is always located thereon.

Furthermore, the method described above and also the advantageous embodiments thereof can also be embodied in a computer program product which comprises a computer-readable medium and a computer program incorporating program code means that is stored thereon. The program code means are suitable for instructing and guiding an operator at the time of implementing the method in accordance with the invention so that he can carry out the steps necessary for the execution of the method in order to detect the position coordinates of all the characteristic points on the pelvis 14 or the adjacent bone parts such as the lumbar vertebrae 64, 66 and 68 that are needed for the calculation of the reference plane 12. The computer-readable medium can, in particular, be a CD or some other form of non volatile storage medium such as a USB memory for example.

What is claimed is:

1. A non-invasive method for the determination of a reference plane of the human pelvis in which method position coordinates of three points A, B, C of the reference plane are determined non-invasively in a reference coordinate system and the reference plane is calculated from the position coordinates of the three points A, B, C, wherein position coordinates of a first point A of the reference plane are determined non-invasively by detecting a first characteristic point (A) on the pelvis, which first characteristic point (A) is located on the reference plane, and wherein position coordinates of a second point (B, C) of the reference plane are determined non-invasively by detecting a second characteristic point (B; C) of the pelvis, which second characteristic point (B; C) is located on the reference plane but not on the median plane of the pelvis, wherein the position of the median plane of the pelvis in the reference coordinate system is determined non-invasively and wherein position coordinates of a third point (C; B) of the reference plane are calculated by mirroring the second point (B; C) of the reference plane on the median plane.

2. The method in accordance with claim 1, wherein the pelvic inlet plane is determined as the reference plane.

3. The method in accordance with claim 1, wherein, for the purposes of non-invasively determining the median plane, position coordinates in the reference system of at least three characteristic points A, D, E, F, G, H, K on the pelvis and/or on bone parts adjacent to the pelvis, which at least three characteristic points A, D, E, F, G, H, K are located on the median plane, are determined by non-invasive detection of the points, and wherein the median plane in the reference system is calculated from the position coordinates of the at least three characteristic points A, D, E, F, G, H, K.

4. The method in accordance with claim 3, wherein one of the at least three characteristic points A, D, E, F, G, H, K on the pelvis is selected in such a way that it is located on both the median plane and on the reference plane.

5. The method in accordance with claim 1, wherein a point of the pelvis which is located as far away as possible from the median plane is selected as the second characteristic point (B; C).

6. The method in accordance with claim 1, wherein the process of detecting the characteristic points A, D, E, F, G, H, K on the pelvis and/or on bone parts adjacent to the pelvis is carried out non-invasively by a percutaneous palpation process thereof using a navigated palpation instrument and/or by ultrasonic scanning using a navigated ultrasonic sensor.

7. The method in accordance with claim 1, wherein a characteristic point on the iliac crest is detected non-invasively as the second characteristic point (B; C).

8. The method in accordance with claim 3, wherein a characteristic point (D, E, F, G, H, K) of the pelvis which is located on the median plane but not on the reference plane is detected non-invasively as one of the at least two further characteristic points D, E, F, G, H, K.

9. The method in accordance with claim 3, wherein a characteristic point (F, G, H) of the lower lumbar spine which is located on the median plane is detected non-invasively as one of the at least three further characteristic points A, D, E, F, G, H, K.

10. The method in accordance with claim 9, wherein at least one tubercle (F, G, H) on one of the first three lumbar vertebrae (64, 66, 68) is detected non-invasively as a characteristic point (F, G, H) of the lower lumbar spine.

11. The method in accordance with claim 3, wherein that point (K) which is located at the centre of a line connecting the two rear upper iliac spines (spina iliaca posterior superior) is selected as one of the at least three further characteristic points A, D, E, F, G, H, K.

12. The method in accordance with claim 11, wherein the position coordinates of the point (K) which is located at the centre of the line connecting the two rear upper iliac spines are calculated from the position coordinates of the two rear upper iliac spines determined by a non-invasive detection process.

13. A device for the non-invasive determination of a reference plane of the human pelvis, comprising a surgical navigation system for determining the position and orientation of at least one surgical device in space incorporating at least one surgical referencing device which comprises at least two marker elements and is arranged on the at least one surgical device or is connectable thereto, incorporating a detection device for detecting radiation emitted by the marker elements or reflected by the marker elements and also incorporating a data processing unit, wherein the actual marker coordinates of positions of the marker elements in space are determinable by the navigation system, wherein position coordinates of three points A, B, C of the reference plane in a reference coordinate system are determinable non-invasively by the device and the reference plane is calculable from the position coordinates of the three points A, B, C by the data processing unit, wherein an instruction is issuable by the device to determine position coordinates of a first point A of the reference plane non-invasively by detecting a first characteristic point A on the pelvis, which first characteristic point A is located on the reference plane, and wherein an instruction is issuable by the device to determine position coordinates of a second point B, C of the reference plane non-invasively by detecting a second characteristic point (B; C) of the pelvis, which second characteristic point (B; C) is located on the reference plane but not on the median plane of the pelvis, wherein the position of the median plane of the pelvis in the reference coordinate system is determinable non-invasively by the device and wherein the data processing unit is configured and programmed such that position coordinates of a third point (C; B) of the reference plane are calculable from the position coordinates of the second point (B; C) of the reference plane by mirroring on the determined median plane.

14. The device in accordance with claim 13, wherein, for the purposes of determining the median plane in a non-invasive manner, one or more instructions are issuable by the device for determining position coordinates of at least three characteristic points A, D, E, F, G, H, K on the pelvis and/or on bone parts adjacent to the pelvis in the reference system by non-invasive detection of the points A, D, E, F, G, H, K, which at least three characteristic points A, D, E, F, G, H, K are located on the median plane, and wherein the data processing unit is configured and programmed such that the median plane in the reference system is calculated from the position coordinates of the at least three characteristic points A, D, E, F, G, H, K.

15. The device in accordance with claim 14, wherein an instruction is issuable thereby that one of the at least three characteristic points A, D, E, F, G, H, K on the pelvis be selected such that it is located on both the median plane and on the reference plane.

16. The device in accordance with claim 13, wherein an instruction is issuable thereby that a point (B; C) of the pelvis which is located as far away as possible from the median plane be selected as the second characteristic point (B; C).

17. The device in accordance with claim 13, further comprising a position detection device with which, in conjunction with the navigation system, position coordinates of the characteristic points A, D, E, F, G, H, K on the pelvis and/or on bone parts adjacent to the pelvis are determinable non-invasively.

18. The device in accordance with claim 13, wherein an instruction is issuable thereby that the pubic bone (symphysis pubis) be detected as the first characteristic point (A).

19. The device in accordance with claim 13, wherein an instruction is issuable thereby that one of the two front upper iliac spines (spina iliaca anterior superior) be detected non-invasively as the second characteristic point (B; C).

20. The device in accordance with claim 14, wherein an instruction is issuable thereby that a characteristic point (D, E, F, G, H, K) of the pelvis which is located on the median plane but not on the reference plane be detected non-invasively as one of the at least two further characteristic points D, E, F, G, H, K.

21. The device in accordance with claim 14, wherein an instruction is issuable thereby that a characteristic point (F, G, H) of the lower lumbar spine which is located on the median plane be detected non-invasively as one of the at least three further characteristic points A, D, E, F, G, H, K.

22. The device in accordance with claim 21, wherein an instruction is issuable thereby that at least one tubercle (F, G, H) on one of the first three lumbar vertebrae be detected non-invasively as a characteristic point (F, G, H) of the lower lumbar spine.

23. The device in accordance with claim 14, wherein it is configured and programmed in such a manner that that point (K) which is located at the centre of a line connecting the two rear upper iliac spines (spina iliaca posterior superior) is virtually determinable as one of the at least three further characteristic points A, D, E, F, G, H, K.

24. A computer program product comprising a computer-readable medium and a computer program stored on the computer-readable medium incorporating program code means which are suitable for implementing a method in accordance with claim 1 when the computer program is running on a computer of a navigation system.

25. A computer program comprising program code means which are suitable for implementing a method in accordance with claim 1 when the computer program is running on a computer of a navigation system.
